(19) United States
(12) Patent Application Publication
(10) Pub. No.: US 2003/0224506 A1
(43) Pub. Date: Dec. 4, 2003

(54) Tiled biochips and the methods of making the same

(75) Inventors: Anoop Agrawal, Tucson, AZ (US); Alan Gordon Goodyear, Tucson, AZ (US); Juan Carlos Lopez Tonazzi, Tucson, AZ (US); Robert S. LeCompte, Tucson, AZ (US)

Correspondence Address: Anoop Agrawal, BIOMICROARRAYS Inc, 4541 East Fort Lowell Road, Tucson, AZ, 85712, US

(73) Assignee: BIOMICROARRAYS INC

(21) Appl. No.: 10/443,217

(22) Filed: May 22, 2003

Related U.S. Application Data
(60) Provisional application No. 60/384,372, filed on May 30, 2002.

(51) Int. Cl. C12M 1/34; H01L 21/00

(52) U.S. Cl. 435/287.2; 438/1

(57) ABSTRACT

This invention describes the advantages of forming integrated biochips including microarrays comprised of tiled assemblies. For a given biochip of this invention, the tiles may have similar or dissimilar properties. Novel, high-speed manufacturing processes are described to assemble such biochips. A preferred embodiment is the use of micro-machined feeders for placing the tiles in the assembly process.

### CROSS REFERENCE TO RELATED APPLICATIONS

This application claims benefit of and priority to U.S. provisional patent application No. 60/384,372 filed May 30, 2002.

### TECHNICAL FIELD

This invention relates to the field of chemistry, molecular biology, biochemistry, medicine and product assembly. The invention is directed to the methods of preparing biochips on a commercial scale with enhanced functionality.

### BACKGROUND OF THE INVENTION

Biochips, including microarrays, for chemical and biochemical analysis have become an important tool in biomedical technology. Biochips allow a high throughput analysis of the products of gene expressions (e.g. polynucleotide, oligonucleotides (e.g., DNA and RNA), proteins, carbohydrates, enzymes, lipids, cells, antibodies, toxins, drugs and others). Several different terms used to describe biochips, e.g., microarrays, DNA chips, DNA arrays, Gene chips, protein chips and the like. A method of making microarrays involves preparing the substrate of a biochip so that it can bind the “probe” molecules. These treatments (generally called “surface activation” or “attachment chemistry”) may involve covalent attachment of linker molecules such as silanes where one end attaches to the biochip substrate (e.g., glass) and the other end is free to chemically or physically bind to the probes. Sometimes multiple linkers may be used for this purpose. The next stage in the process is to deposit a spatial array of probe molecules. Generally, this is done in two ways. In one method for analyzing DNA fragments, the probes are sequentially synthesized on the substrate by introducing one or more nucleotide base at a time (U.S. Pat. No. 5,744,305; WO 00/61282; U.S. Pat. Nos. 5,700,637; 6,545,758 and 6,375,903) that link to form a single strand oligonucleotide molecule. The sequence of oligonucleotide is controlled for each array element. In another method, probes are dispensed as spots mainly using aqueous solutions. The probes in solution bind to the substrate as the water or any other carrier fluid evaporates or is removed. Any unbound probe is then washed away. In either case an array of elements of known probes at known locations is formed on the substrate with each array element being spatially separated from the next. These array elements may be arranged in a row or in a two dimensional matrix with rows and columns. This array of probes is then subjected to a solution containing unknown sample of biomolecules (the “target” molecules). These targets then bind only to the specific probes dictated by their chemistry. As an example, if the probes comprise of single chain oligonucleotides (DNA fragments) and the target is comprised of unknown single fragments of DNA then only complementary targets would find a matching probe with which it will bind or hybridize. Depending on the extent of the match, the concentration of the target being trapped at a particular probe site will be different. This process is called hybridization. This binding is detected by a variety of means and then, using statistics, the extent of binding at different sites is correlated and the identity of the unknown sample revealed. To detect binding several techniques may be used. For example, the target molecules may be tagged with detectable materials such as fluorescent molecules, radioactive atoms or metal nano-particles, which are then detected in an appropriate scanner, or the attached molecules are detached using lasers in each array element sequentially which are then observed on a mass spectrometer. However other arrays may be made for proteins where capture antibodies may be used as probes. The detectable moieties may also be attached later to the hybridized targets. In a similar fashion, other arrays may be made for chemicals and other biological materials. Details on biochip technology are given in several books such as by Schena and by Gibson et. al. In all cases, the substrate is a single continuous entity.

In an alternative method, microarrays may be made by assembling particulate carriers or tiles-like entities (“tiles”) carrying these reactive probes onto the underlying substrate surface (the “US S”). In this process, tiles carrying reactive probe materials are set onto a substrate. This is described in US patent application 2001/0039072 by Nagasawa et. al. Attaching the probes to the tiles and then attaching the tile to the USS may offer advantages over the conventional processes described earlier, of attaching different probes to a single, continuous microarray surface. The tiles may be porous or solid and the substrate may have compartments which contain these tiles. A similar concept is described in PCT application WO 02/02794 to Wei et. al, where tiles are placed in distinct, individual compartments on a substrate. Here each tile may be an array element of the microarray or a microarray with several array elements. However, the above arrays could be very laborious to fabricate, particularly if many chips have to be assembled.

U.S. patent application Ser. No. 10/291,467 filed on Nov. 8, 2002 describes microarrays, where the same substrate exhibits different regions that may possess different properties. An example of this is shown in FIG. 1a. where a microarray glass slide 110 (75 mm×25 mm in size) shows eight distinct regions as shown by 111. These differences may be due to differences in surface activation, differences in the type of probes, or different surface properties, etc. This was done to increase the dynamic range of the microarrays. The microarray has distinguishable regions with different sensitivities or may detect different types of materials in different areas, and even be able to detect using a different mechanism in these areas to achieve any of the above purposes. However, according to the present invention these different regions would be individual tiles that may be assembled on a substrate in a high speed and economical fashion.

Microarrays have also been suggested where the particulate carriers can be beads that are placed on substrates, where each bead is made distinct from the others by having different probes attached to it. Such microarrays are described in PCT application WO 00/61198 and in the US patent application 2003/0044801. These beads are placed in grooved cavities which are pre-formed on a substrate without locking them in place. The beads touch several neighbors and are free to move within these cavities. While the focus of the present invention is mainly on tiles which are anchored on to the underlying surface, the bead microarrays of the prior art will also benefit from the assembly processes described in this invention. Planarity of the substrate (USS) is defined relative to the tile or the bead size. If for example, the USS surface roughness is smaller than the tile (or bead) size then it is considered planar. Bead size is generally defined as average diameter of the bead, and the tile size for this issue is defined as any of its average dimension such as length, width and height.

Additionally, the microarrays may also be formed on flexible substrates. As an example, one may have a an underlying substrate in a tape format where the tape may be wound in a cassette format. Tiles, preferably made of flexible materials, may be placed on such flexible substrates to form the microarray. For processing the array, the tape may be transferred from one reel to the other in order to expose the surface. In hybridization process, for example, the tape may also be passed from one reel to the next through a hybridization solution or the entire cassette may be placed in the hybridization solution as long as the tape is loosely wrapped or uses spacers to allow the target-bearing fluids to penetrate and interact.

Another kind of biochips are based on micro-fluidic devices. In this case the substrate has reservoirs, connecting channels, valves, reaction chambers etc., which are micro fabricated on the substrate. The construction is such as to enable fluids to move mix and interact at different spatial locations on the substrate surface (see Kopf-Sill, A. R. et al). Individual tiles exhibiting micro-fluidic features may be assembled on a USS using this invention. The microfeatures may be in a communicating relationship from one tile to the next, or each tile may comprise of a self contained micro-fluidic device.

The purpose of this invention is to describe novel methods to mass-produce tiled biochips at an attractive cost, and which result in enhanced functionality that was not practical to date. These tiled biochips may exhibit distinct attachment characteristics and different material properties.

### SUMMARY OF INVENTION

This invention describes a novel approach to assemble tiles to yield integrated biochips. The individual tiles may be fabricated from a larger single entity. Generally, these tiles will have a surface treatment that is able to bind probe molecules. The tiles may have a variety of shapes that include but are not limited to flat tile-like shapes or bead-like objects of varying porosity. A collection of different tiles is then brought together to form an integrated assembly. In one preferred embodiment, the tiles are combined onto a surface of underlying substrate (“USS”) to yield the final product, preferably by placing and anchoring them on the USS. These tiles may be sized so that each is a separate and distinct element of a microarray, or each tile may itself comprise of several distinct array elements. Generally, the tiles may have different surfaces characteristics and different surface attachment chemistries. The probes may be deposited using conventional means onto the integrated biochip or specifically in this case integrated microarray assembly (“IMA”). The differences in surface properties may arise because the tiles are made from different materials or have different surface microstructures. Additionally, they may have different surface activation, different functions or combinations of these properties. A preferred assembly method for these tiles uses a high-speed robotic assembly process similar to that widely practiced in the mechanical assembly of electronic components, such as surface mount components, on a printed circuit board (PCB). Another preferred assembly to place tiles at high rates will use micromachines.

A further preferred assembly method involves a multi-step process whereby an adhesive is introduced to a specific, known location or locations on the USS. A plurality of tiles is brought into contact with the adhesive regions wherein some adhere to the surface of the USS. A subsequent process removes the excess and non-sticking tiles from the USS. The process is repeated until the USS is populated with tiles at known locations. In this preferred method, the tiles may also have the probes attached to their surfaces.

### DESCRIPTION OF THE FIGURES

FIG. 1a: A microarray biochip with several different regions.

FIG. 1b: Shows schematics of a microarray assembly made by this invention.

FIG. 2: A MEMS micro-machine dispenser showing beads being dispensed.

FIG. 3: Horizontal configuration of MEMS micro-machine dispensers.

FIG. 4: Vertical configuration of MEMS micro-machine dispensers.

FIG. 5: A MEMS micro-machine dispenser with several feeds.

FIG. 6: A stack of MEMS micro-machine dispensers.

FIG. 7: A MEMS micro-machine dispenser showing tiles being dispensed.

### DETAILED DESCRIPTION OF THE INVENTION

One objective of this invention is the formation of tiles for use in biochips and chemical analysis with different attributes and also methods of making them in production quantities using efficient processes.

Another objective of this invention is to make integrated biochips and applications of such biochips where the various elements of the biochips are tiles, which may have varying characteristics, which are assembled on to an underlying surface substrate (“USS”) yielding a monolithic assembly.

Another objective of this invention is to form integrated biochips with enhanced functionality including chips with high dynamic range and enable new applications for biochips.

Yet another objective of this invention is to enable the fabrication of integrated biochip assemblies using high speed assembly processes.

Tile Characteristics and the Methods of Making Tiles

In a conventional microarray biochip, the array elements (comprising the microarray) are generally formed on the surface in two ways. Both methods, however, require the activation of the surface with a linker molecule that allows the probe molecule to become attached, and therefore localized, to the surface. In the first method, a probe is deposited at a location on the activated surface to yield an array element. Typically, spots (array elements) in a size of 50 to 500 microns are deposited in an array format using different solutions for each probe that is different. Because of differential drying, the spot morphology may vary substantially from one spot to the next. In the second method, probes are synthesized using monomeric building blocks added sequentially to the first building block that it anchored to the substrate surface by a linker molecule. In either case probes are deposited in each array element independent of each other. In the method of this invention, any of the above methods could be practiced to deposit the probes, however, it also enables other methods that are cost effective. A large wafer may be used to attach the probes uniformly, which is then diced into tiles which are then used to assemble microarrays of this invention. Since surface activation or probe attachment is done on large scale which may comprise tens, hundreds or even thousands of tiles, the process becomes more reproducible from tile to tile, and further, the processes may be tuned to have optimized process to get the maximum benefit from that type of tile. For example the process used in U.S. Pat. No. 5,744,305 can be used to synthesize the oligonucleotide probe without masks so that the same probe is attached all over the wafer surface. “Activation” of surface in this invention is defined as a step where a treatment is done on a substrate to facilitate probe binding. If there are a series of chemical or physical treatments done to a substrate before probe binding then any of these is activation.

Tiles, may be processed by several methods for activation of the surface for probe deposition. In a large scale method, a wafer or large substrate may be spin coated, dip coated, vapor deposited, sprayed, meniscus coated, synthesized or any other high speed means with either or both of the linker molecules followed by the probes (including oligonucleotides). The wafers or large substrates may even have pre-marked lines that depict the tiles. Any border regions associated with the parting of the tiles may be optionally coated with materials (or be surface treated) so that the probe does not adhere in these areas, or it may be washed away from these areas in subsequent processing. The surface of the large substrate or wafer may even be covered with a protective film before dicing into tiles. The coating protects the surface during the assembly so that the surface is not damaged or altered since the parts may be picked up pneumatically by holding on to the tile surface. In some cases, mechanical edge gripping of the tile may be preferable since the surface would be not directly accessed. This film may be removed after the assembly of the tiles is completed or by the end-user of the integrated microarray. A preferred method of removal is by washing these in water where the film either dissolves or lifts-off. Some preferred water soluble, film-forming materials are polyvinvl alcohol, polyacrylamide, poly-ethers and co-polymers comprising these. The USS can be of any desirable shape, e.g., round, square, rectangular, ribbon shaped, etc. In one preferred embodiment this may be made of a fabric, polymer sheet (e.g., polyamide, polyacrylamide, nitrocellulose), micro-sheet glass or composites such as reinforced sheets (e.g., polyacrylamide on a polyester backing) and metal-coated glass and polymers, etc. In ribbons, linker system or probe may be attached to the surface using a roll-to-roll type processing similar to the process used in coatings and the printing industry. The ribbon may be cut into tiles at the assembly station. Each individual tile is generally smaller in footprint as compared to an integrated microarray assembly. These tiles may also be small in size and may be down to 0.1 cm×0.1 cm or smaller. The preferred thickness of these will be generally smaller or equal to their width or length. The individual tiles may even have shapes other than planar and may be porous. For tiles less than 0.1 cm×0.1 cm, for example, the preferred shape may be bead or bead-like that are also solid or porous.

For microarray application the tiles may be sized so that each represents only one array element, or one array element is made up of several tiles, or each tile has more than one array element i.e., each tile being a microarray. Each tile may comprise of a different material with different characteristics and may also have different surface activation coatings so that the probes that become bound to each of the tiles may provide a different functionality. This approach is advantageous in building protein arrays. The protein concentrations in a biological sample varies several orders of magnitude compared to mRNA levels (see Mitchell, P., and in Zhu, H., et. al.,). So protein measurement systems should be able to provide several orders of magnitude larger dynamic range when compared to mRNA measurements. One way to overcome this problem is to have different attachment antibodies on different tiles so that one antibody may bind specifically to a rare protein and the other antibody to a more abundant protein. Careful selection of the relative surface properties of the tiles and the different characteristics and concentration of the different antibody attachments will result in a protein array that can detect a wider set of protein interactions on a single integrated array. More on the arrays of protein-capture agents is also described in U.S. Pat. No. 6,329,209 where array elements are fabricated with different probes or capture agents for proteins. This concept is not different from any of the current microarrays where each array element with a distinct probe is formed separately on a substrate.

The tiling method of this invention provides yet another useful functionality: for example, each tile may have a series of spots (or array elements) constituting different capture agents even if the chemical nature of these is same from one tile to the next. However, each tile may have a different surface morphology or a surface treatment so that it provides different densities of capture agents. In this fashion a more abundant protein may saturate the signal from a high surface density tile, but one will be able to see proteins which are rarer on a linear scale, where as in low surface area, the highly prevalent proteins will be observed within a linear scale, while the signal for rare proteins may be below the detection threshold. A series of increasing gain (surface area) tiles may provide calibration of signals from one to the next, and result in a biochip with a wider linear range. This concept may also be used in DNA and mRNA microarrays to get better overall linearity. Surface morphology may also be changed as described below. Methods to make micro-structured surfaces are given in U.S. patent application Ser. No. 10/291,467 filed on Nov. 8, 2002, which is incorporated herein by reference. These microstructured or “textured” surfaces result in increased area because of the availability of the 3^{rd} dimension as compared to planar surfaces. Porous coatings on tiles or porous tiles may also be used to increase the surface area. Concept related to porous coatings for microarrays are described in PCT application WO 00/61282, US patent applications 2003/0003474 and 2003/0036085.

The tiles may even have different activation chemistries. For example an mRNA or a DNA array may have tiles with different surface chemistries for activation. Some tiles may be activated with one type of silane and the others with another type. Specifically, there may be tiles with an epoxy silane so that probes can be put down which are conducive to this chemistry, and the other tiles may have amino silane chemistry for attachment of different kind of probes. The integrated biochip for microarray application will be referred to as integrated microarray assembly (“IMA”). IMA can then be used to simultaneously hybridize a wider set of molecules from the incoming target material. One may use the same silane or different silanes but vary their surface density on the different tiles. Methods to vary surface density may be found in publication by Kim, J. H., et al, and in publication by Oh, S. J. Some of the other properties that may distinguish the performance of the tiles are, but are not limited to, differences in porosity, in surface iso-electric potential, probe densities, probe types (e.g., oligo or poly nucleotide sequences) and differences in other physical and/or chemical characteristics. The tiles may also be activated by other chemical processes which change the surface chemistry. These may also be based on plasma treatments that may use reactive gases such as ammonia and oxygen.

It is not necessary that all of the tiles for a given IMA be made of the same material, e.g., for some, borosilicate glass may be used, for others soda lime glass may be used and yet for others metal or polymers may be used. Some may be coated with cross-linked polymers to give three dimensional attachment sites. Yet others may be coated with porous gel-like coatings. The size and shape of the various tiles in one microarray assembly may be different. This flexibility of combining various materials, shapes and sizes of tiles for microarray application is novel. This may be of significant advantage where array elements on the IMA can be analyzed by different means. Analysis of some tiles may be by optical means (e.g., by fluorescent probes), and other tiles by radioactive methods, while yet others use chemiluminescence, and some may be analyzed by laser desorption (e.g., by mass spectrometer), and yet others by an electronic measurement. This can help in detecting same types of materials by different methods for better statistical confirmation, extending the dynamic range or for any other reason where different methods of analysis on the same micro-array provide more information on a single test platform. This further extends the functionality and the flexibility of microarrays and opens possibilities for new applications

These tiles may even have different analytical functions or “utility” in the sense that some of these may be for DNA analysis and the others for protein analysis. These tiles may have protective coatings. The protective coatings on each individual tile may be designed in a way so that they are removed in a media when a particular area of the assembly is processed, such as hybridization, washing, etc. Alternatively, the protective coatings in different regions may be so designed that they come-off in different medias (e.g. solvents and solutions). As explained earlier, each individual array element assembled together itself may be a different tile, or each tile which is assembled together may be a microarray having several array elements. The protective coating described above may also be used to encapsulate a media to keep the probes in a desired state during storage. For example some of the protein probes may require an aqueous medium to keep them from denaturing. The protective layer can be a multilayer film where the inner layer facing the probe is hydrated (e.g., polyacrylamide, polyvinyl alcohol, etc.), and the outer layer is impervious to water (polyester, polyvinylidene chloride based copolymers, etc.). Some examples of encapsulated hydration layers on the surface are described in U.S. Pat. No. 5,216,536.

The above concepts are not limited to biochips used in microarray analysis. Biochips using principles of microfluidics may also be fabricated by this invention. These may also have tiles which have embedded reaction chambers, communicating channels, reservoirs, etc., or other microfluidic techniques. Details of several types of microdevices which can be used in this invention are for example described in a publication by Heller, M. J., et al. These devices are also called “lab on a chip”. The size of any features such as reservoirs are less than 100 microliters and width and depth of channels are usually less than 1000 micrometers. Such devices can be put on individual tiles and then assembled together as described in this invention. This invention provides the versatility of integrating many different functions in one device that was not available before. This invention permits the large scale fabrication of these small microfluidic or micro-electromechanical systems (MEMS) devices and their subsequent assembly. Mems relates mainly to use of semiconductor processing techniques to create small parts in silicon or other compatible materials. More on this is given in the section below.

The spacing between the tiles may be adjusted so that microchannels are formed that permit the flow of various fluids within, across or through the surface of the microarray. Further, the placement of tiles on a first USS followed by repeating this using second USS on top of the tiles of the first array will facilitate other microfluidic devices. This aspect is not limited in terms of the number of layers.

Assembly of Tiles

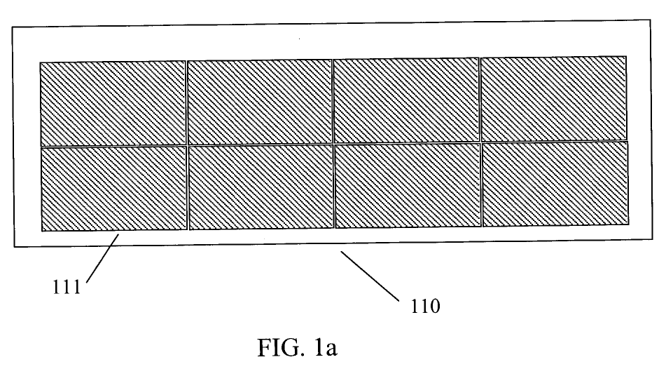

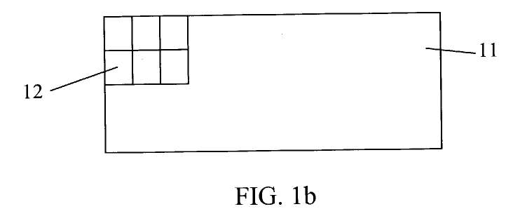

In the assembly process these tiles may be bonded to each other, and/or they may be bonded on to a USS. In the latter case, the USS may be removed or it may become part of the finished microarray. FIG. 1b shows schematics of a partially-completed microarray assembly made by this invention. The USS is shown as 11 and the tiles as 12. In one method, an adhesive may be uniformly applied (or locally applied by means of a mask) to the USS before the assembly process so that its surface may temporarily hold the tiles in place. After depositing all the tiles, pressure and temperature may be applied so as the adhesive is squeezed and all the array elements are pressed down to a uniform plane. The temperature itself may cure the adhesive, or a thermoplastic adhesive may solidify after cooling. One may also use a radiative method (e.g., UV, IR, microwave or nuclear radiation) to cure this material. Solders, surface tension, placeholders, indentations, magnetic attraction, electric field interactions, mechanical locks, or other methods may be also used to keep individual tiles in place. As an example, if beads or bead-like elements are used as tiles, one may have spherical sections molded or carved in the USS so that the beads or bead-like elements could self-center on a location. In an alternative method, the adhesive may be applied to each tile and/or to the USS locally during the process. After the tile is placed, the adhesive is cured using localized heat or radiation (ultraviolet, visible, infra-red and microwaves). The process is repeated till the assembly is complete.

USS may be metallic (e.g., silicon and aluminum), polymeric (e.g., nylon and polycarbonate) or inorganic such as a ceramic, glass and quartz. This may be molded out of polymers and glasses, or be cut from extruded or floated sheets. This may even have a coating or a stack of coatings (e.g., a light reflector to enhance the light activation process or to reduce background signal by interference or blocking). They may even have marks and lines to align the tiles on its surface using optical vision (e.g. camera systems). In some applications, it may be appropriate to mark the tiles with an identifying feature that is recognized by the assembly machine during assembly or by the analyzer (such as fluorescent scanner) during analysis of the integrated biochip. This may be a bar code or another simple mechanical feature. Other forms of identification may be used such as, but not limited to, materials with varying signatures that can be detected and correlated to location.

In another method, an adhesive material is applied to the surface of the USS by using a robotic process at distinct and known locations. The tiles, but in particular beads and bead-like structures, are introduced to these locations in a timely fashion. These may be introduced via a fluid (e.g. gas) stream or direct dispensing. Contact between the tiles and the tacky adhesive causes the tiles to become attached where there is adhesive on the USS. The adhesive is permitted to cure so that it is no longer a location to which more tiles can adhere. This curing may be activated with heat, light (UV, visible and infrared) or other radiation sources. Excess, non-sticking or loose tiles are subsequently removed. The removal may be by a blowing action of a gas such as air, nitrogen, argon, or by vacuum or mechanically (e.g. by inclining the substrate). The process is then repeated at different locations on the USS until the IMA is fully and appropriately populated with array elements. In this method, the tiles may have only activated surfaces or they may have probes attached prior to assembly onto the USS. This method may also offer advantages for the attachment of small tiles where rigorous alignment and spacing of the tiles is not critical. Based on the design constraints, i.e., size of the tiles and the size of adhesive spots, one or more tiles may stick to each location. Typically, each location is a microarray element.

In some applications it may be appropriate to stack several tiles on top of or in close proximity of each other. However, in this case, it must be ensured that the tiles lower in the stack are accessible to the targets, such as by porosity, channels or physical separators and are accessible for measurement. In some applications, it may be appropriate to stack the tiles in an overlapping mode in a manner similar to that used with roof tiles. Additionally, it may be appropriate to alter the size of the tile as the stack height increase to produce a pyramid like structure or micro-feature.

Tiles that provide various types of utility may be combined on to one USS. Some of the tiles may have an active or passive electrical or electronic function. This may include, but not be limited to, the emission of electromagnetic radiation and the subsequent detection of re-radiated signals emanating from molecules attached to the same or other tiles on the USS. Some tiles may be used for depositing the microarray or another kind of a biochip function, and yet other tiles may provide other functions such as optical coupling, lensing, detection elements, or electronics etc.

Assembly is preferably done by high-speed robotic machines similar to those used in electronics assembly plants (e.g. see Chip placement and multi-function mounting machines from Fuji Machine Manufacturing Co (Japan)). The tiles are preferably bonded or firmly retained on to the USS to form an IMA. A general description of the technology is given in a book by Rowland, R. J. and Marcoux, P. It is novel to use such procedures to assemble microarrays for chemical and biological analysis. In general, the machine consists of a feed system that typically delivers components into a placement head that in turn, delivers said components to the printed circuit board (PCB). The placement head may have all the motions, such as x-y-z and rotation, or the conveyor for the PCB may provide the x-y motion and the other motions are provided by the placement head. Generally, to facilitate this invention, the most modifications will be required in the feed mechanism and the placement head or heads. This is because the size and handling requirements of the tiles associated with this invention. Specific examples of the placement heads for tiles will be described later. The assembly method described in this invention may also be used to assemble products suggested by Nagasawa (US 2001/0039072) and Wei (WO 02/02794) at high speeds and economically.

PCT application WO 02/02794 by Wei describes an integrated microarray where individual arrays are placed in distinct reaction wells called micro-locations. This has use only when a biochip has to be used in a mode where distinct reaction wells are required. As such, this invention requires the target material in one reaction well to be kept separate from that in another reaction well. In our invention, we can have tiles, with varying characteristics, assembled inside the reaction wells or surface mounted. Further the design of the IMA may be such so that some tiles are mounted in the reaction wells and yet others are on the surface of the IMA (i.e. on the USS but outside of any reaction well).

Another variation of the invention is combination of electrical or electronic modules (or “electronic tiles”) and microarray tiles for an active integrated microarray assembly (“AIMA”) i.e., both electronic and analytical tiles may be assembled together on the same substrate (both sides of the USS are available for use) providing increased utility. Additionally the USS may have a plurality of layers that facilitate these interactions. The layers may be interconnected. Such elements may be interconnected by various means to facilitate communication and analysis. If necessary different placement heads can be used for each of the components on the same machine. These tiles are in a communicative relationship to each other. These tiles may be fabricated and placed so that they communicate by taking advantage of connectors and methods used in typical electronic and optical surface mounted integrated chips, some of these features are—use of both sides of the substrates, using ball grid array connectors, solder pads, optical interconnects, etc. IMA's or AIMA's may also be assembled on to a conventional printed circuit board, or other electronic substrate, along with the other components to form an interactive circuit. The circuitry and the input/output devices and connectors on this interactive circuit board may provide signal detection, signal analysis, analytic capacity, probing and data storage protocols, data processing and communication with other equipment. Some examples of electronic components assembled along with the biochips are microprocessors, displays, light emitters, light detectors, and memory storage devices on board to provide more functionality to the integrated chip. Some examples of optical components are light filters, waveguides, lenses, collimators, switches, etc. Some of the Electronic tiles may be held by a snap-on or other quick release type of a design so that they are replaceable in the field. In other forms, the probe-bearing tile may be held by a snap-on or other quick release device so that different probe bearing tiles can be loaded to facilitate rapid analysis of target material. In a preferred embodiment, a number of probe-bearing tiles could compared and contrasted for rapid detection of small difference in target performance. In another variation each of the assembled chips could be based on planar wave-guides (e.g. see publication by Ehrat, M.,) and the AIMA functions as a motherboard, which guides both the excitation light signal and the emitted light signal to and from the various tiles mounted on it.

The assembly process itself may be configured in a number of ways. In one method the tiles are placed in turret feeds or be placed on reels (or tapes). These are mounted on “pick and place” robotic machines such as Fuji CP-732E machine or equivalent. Clearly, these machines are designed for electronics assembly and will have to be modified for use in the assembly process for this invention. In a fashion similar to that of electronic component feeders, the tiles are secured on tapes that are wound in to reels that are fixed to the assembly machine at distinct feeder locations grouped into stations. The machine has information that identifies which tile is at which feeder location. Each feeder location may contain up to thousands of tiles. On each assembly machine, there may be more than one station. In other instances, single reels may contain predetermined, different sequences of tiles.

Additionally, the tiles can be located in hoppers, bins or screw feeders that deliver the tile to the placement head or heads via either pneumatic or fluidic techniques.

There may be several similar stations such that, once the feedstock in a particular station is exhausted, and second station assumes the function of placement of that particular tile, while the depleted station that is removed and replenished.

This invention requires that there be at least two tiles on an IMA or an AIMA. The placement of distinct tiles by robotic means will become either difficult or uneconomical when the size of the tile reaches 0.1 cm×0.1 cm. For tiles with sizes less than this limit, the preferred placement method involves tiles that are bead or bead-like placed into distinct areas of adhesive located on the USS. This technique has been previously described elsewhere in this invention. It must be emphasized that the mechanical placement and the fluidic placement are embodiments of the same invention. For economic reasons, the preferred speeds of mechanically assembling tiles should exceed 100 tiles/hour and more preferably in excess of 10,000 tiles/hour and most preferably in excess of 500,000 tiles/hour. These speeds relate to each placement head. There may be more than one placement head on a machine. The upper limit will be dictated by the concentration of bead and bead-like tiles in the fluidic or pneumatic assembly process. Both assembly techniques should be able to handle multiple feed stations, preferably greater than 10 feed stations, more preferably greater than 100 feeds stations and most preferably greater than 1,000 feed stations. For an IMA or an AIMA assembled to a 75×25 mm format USS surface, several of these USS may be placed together and assembled simultaneously. Alternatively, one may also use a large USS and then assemble all the elements on this and then dice this larger USS to a size of a standard format microarray (i.e. 75×25 mm). The machine may assemble all of the array elements, or only a few before the USS is moved to another machine where a different set of tiles is mounted and so on until the process is complete. Since the number of tiles on each IMA or AIMA may range from more than one to several hundred thousand or more, it may be necessary to use more than one machine to assemble parts efficiently as the number of components increase. In another alternative several sets of tiles may be pre-assembled in turrets or tapes in a sequence so that fewer feeds are required.

If the tiles are being supplied in a tape form, one may attach the analyte on to the tiles just prior to the assembly, e.g., the tape may pass through an analyte solution followed by drying or curing and then applying a protective layer by dipping, spraying, etc.

Typically the feeder tapes consist of a bottom layer and a top layer with the components sandwiched between them. Additionally, tapes that secure the edges of the tile are used. In the latter case, the top layer is peeled, and the components are picked up from the bottom layer. The bottom layer itself may have molded pockets for each component. It may even have multiple pockets for each set of components where the complete set is picked up and placed on the USS in a single step.

Conventional pick and place component assembly machines are limited to components of a particular size. Difficulties associated with capture of the components and subsequent accurate repeatable placements have resulted an effective limit in region of 0.1×0.1 mm. Additionally the speed of placement must be increased in order to maintain productivity. Some examples of alternative design of placement heads suitable for placing small tile or tile-like objects (generally less than 100 microns in size) at large rates are shown below. Robotic placement heads described above and the ones described below based on micro-electromechanical systems are called “placement systems” in this invention.

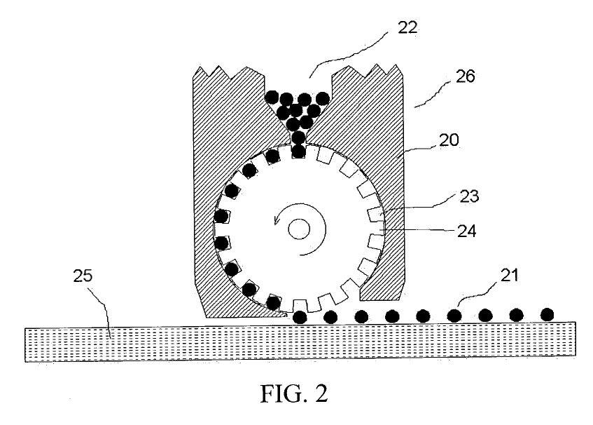

The tiles may be placed at large rates using placement heads that make use of micro-machines (also called micro-electro-mechanical systems or MEMS). Many of the figures below show the tiles in the shape of beads, but any of these may be adapted for other shapes with simple changes as illustrated in one of the examples below. An example of a placement head utilizing a micro-machine is shown in FIG. 2. Since these are very compact, the placement head may accommodate from tens to thousands of these without becoming too bulky. MEMS are increasingly utilized in optical communication industry to make moving components such as mirrors and other devices. References on making MEMS can be found in several places, e.g., see MEMS Handbook by Gad-el-Hak, M. Micromachining for this disclosure is described as elements formed on a substrate that have motion. Further, each micromachine may have several components, such as feed channels, hoppers, dispensing wheels, etc., and each distinct feature is smaller than 5 mm, and more preferably smaller than 1 mm.

Micro-electromechanical systems (MEMS) is a technology that combines tiny mechanical devices such as sensors, valves, gears, mirrors, and actuators embedded in semiconductor chips. Further, these are partially or fully manufactured using microfabrication techniques used in the semiconductor industry. The size of the devices produced by MEMS technology can be any which will be usable in the application. Devices as large as 5 cm in size to 10 nm in size have been fabricated. In this application the practical limits at present are around 0.1 micrometer. MEMS devices have been realized in silicon based technology, largely borrowed from microelectronics technology. However, in recent years a variety of other materials have been used to create MEMS devices, including polymers, ceramic, GaAs, SiC and plated metals.

FIG. 2 shows a part of the placement head 26, which is able to dispense a row of tiles 21 onto a substrate 25. In this embodiment, it is shown that the placement head moves relative to the substrate. In this part of the placement head, the feeder hopper 22, the wheel dispenser 24 are all made by micromachining a silicon wafer 20. The depth of machining is generally less than the thickness of the silicon wafer. To enclose this structure a cover plate can be provided (not shown) which may be made of any suitable material, such as another silicon wafer, glass, metal, polymer, etc. Silicon is a preferred material for micromachining given the state of technology to micromachine this material, however any other suitable material can also be used. The depth of the grooves 23 in the wheel 24 can be customized based on the size of the beads or tiles. It is shown that each groove holds one tile, however, systems can be designed to hold more than one tile. The number of grooves in the wheel can be any which will get the processing accomplished, in, one preferred embodiment this number is from one to ten. The feeding of the tiles in the dispenser, and dispensing of the tiles onto the substrate may be aided pneumatically, by surface tension or viscous drag of a liquid on the substrate, etc.

To ensure that each of the groove is loaded with tile(s) one may design a light source and a photodetector (not shown) to check the presence or absence of a tile in every groove as it passes a fixed spot. These may be mounted external to the MEMS structure, such as on the cover plate or preferably designed within the same silicon block. It is preferred to make both MEMS and other electronic or optical components within the same material block using standard semiconductor processing principles. These could include connections to power micromachine, controllers, optical encoders/decoders, electronic vibrators for the hopper using piezoelectric coatings, etc. The hoppers themselves could be continuously fed from pneumatic tubes connected to reservoirs preferably placed off the placement head to keep its mass low. Alternatively, when the hopper is exhausted, the machine stops and either the placement head travels to another location for a refill or waits for an operator intervention.

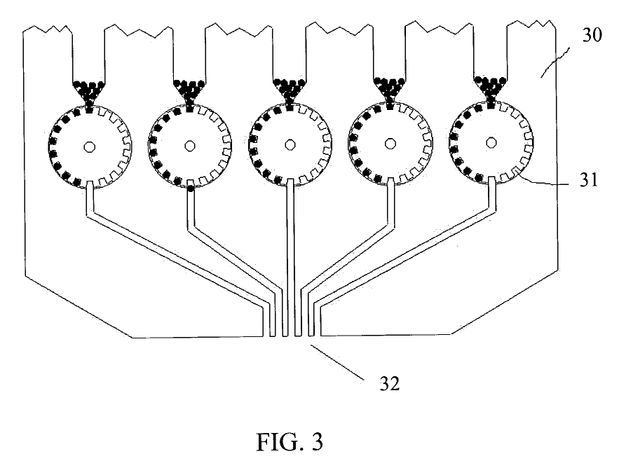

It would be preferable that the placement head feeds a number of rows simultaneously so that the throughput can be increased. Each row may feed different type of tiles based on chemistry, size, or any other characteristics. FIG. 3 shows an embodiment where several dispensers 31 are machined in a single silicon wafer 30 arranged in a horizontal placement. These dispense in a area 32 so that the spacing can be easily controlled between the various rows. Depending on the requirements tens, hundreds or even thousands of such machines can be fabricated on a single wafer. All of these can share as much of the common circuitry as possible. The back surface (not shown) of the wafer can be primarily used for electronic circuitry placement and may be connected through vias on the front surface where the MEMS structures are located. As an example, a placement head containing this kind of wafer with 100 feeders, and each feeder dispensing at a rate of 1 tile/second, can achieve rates of 360,000 tiles/hour. Several of such wafers are combined to get even a more spectacular performance. One of the ways to do this is described below. It is easy to see that the limitations on the number of feeds and the rate can be overcome as compared to in the electronics assembly.

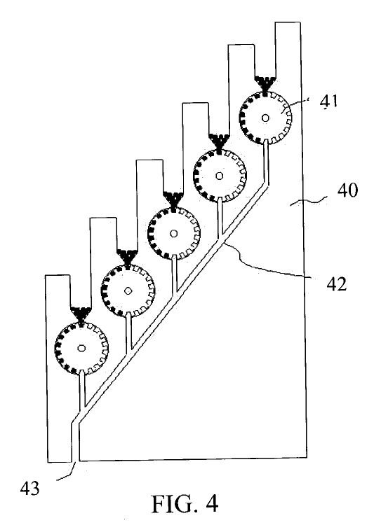

FIG. 4 shows yet another embodiment where several of the dispensers 41 are arranged in a silicon wafer 40 in a different way, called vertical placement. All these feed in one chute 42 and dispense at one point 43. Depending on the timing and the speed of rotation of different dispensers, the sequence of tiles being dispensed can be controlled (assuming that each dispenser is being fed by different kind of beads). One may combine both the vertical placement and the horizontal placement as described earlier to make a placement head that may deliver thousands of different tiles simultaneously in a known sequence.

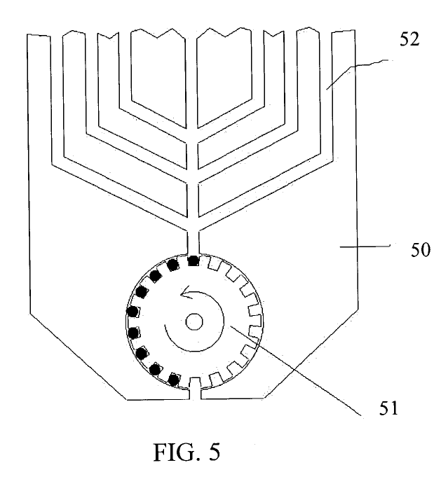

FIG. 5 shows another arrangement to achieve dispensing a single point with a predetermined tile sequence. The silicon block (or wafer) 50 shows one dispenser 51 that is connected to a number of feeds 52. These feeds are connected to various types of tile feedstock. Each of which may even be fed at a controlled rate by other dispensers (not shown) that are machined in the same block.

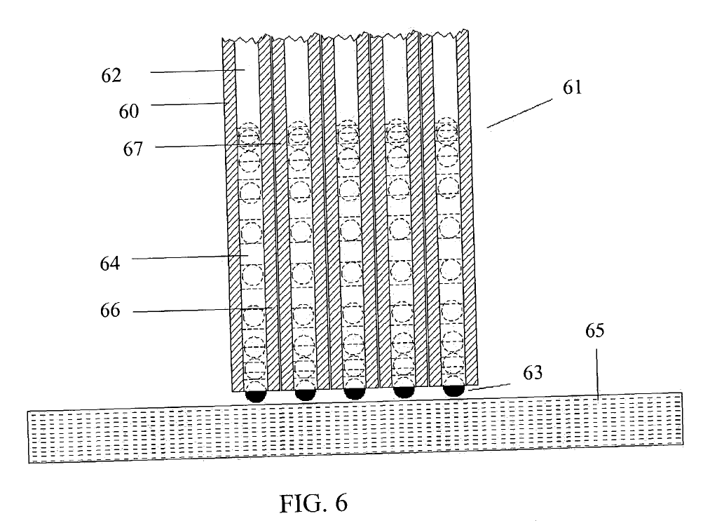

FIG. 6 shows a side view where several of the silicon wafers may be combined together to yield a 3-dimesional block 61. The block consists of several dispensers assembled together. For example one of the dispensers consists of the wafer 60 in which the feeder area 62 and the wheel dispenser 64 are fabricated. The cover plate 66 is optional between the dispensers, as the backside of the next wafer 67 may also act as cover. The beads 63 are dispensed on the substrate 65. This shows only one dispenser in each wafer, however, several dispensers can be combined in each plane as discussed in the vertical and the horizontal placement concept. The block may even be so sized that all beads to produce a single or multiple microarray are delivered in one shot.

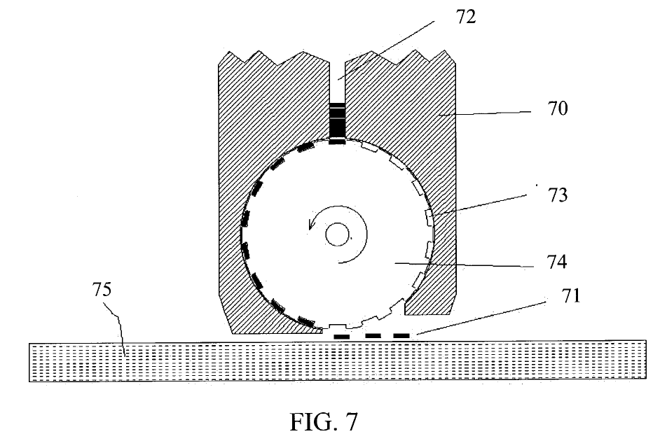

The concepts described as above show tiles as beads, but other shapes such as planar tiles may also be used. A drawing that shows this more clearly is shown in FIG. 7. The silicon block 70 has a feeder 72 for the tiles that are caught in the grooves 73 of the wheel dispenser 74 and the tiles 71 are dispensed on the substrate 75. The placement heads may combine beads, flat tiles and other shapes to be deposited on one microarray assembly. Such systems may also be used to dispense liquids so as to form arrays on a substrate by dispensing probes from solutions and then drying the solvents.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

### REFERENCES

### Patents

U.S. Pat. No. 5,744,305

WO 00/61282

U.S. Pat. No. 5,700,637

U.S. Pat. No. 6,545,758

U.S. Pat. No. 6,375,903

US published application 2001/0039072 to Nagasawa et. al.

WO 02/02794 to Wei, et. al.

U.S. application Ser. No. 10/291,467

WO 00/61198

US published application 2003/0044801

U.S. Pat. No. 6,329,209

US published application 2003/0003474

US published application 2003/0036085

U.S. Pat. No. 5,216,536

### Non-Patented Literature

Mark Schena, ed., Microarray Biochip Technology, Eaton Publishing, Natick, Mass., (2000).

Greg Gibson and Spencer Muse, A Primer in Genome Science, Sinauer Associates, Sunderland, Mass., (2002).

Kopf-Sill, A. R. et al, Micro and nano fabricated electro-optical mechanical systems for biomedical and environmental applications, SPIE proceedings 2978, p-172 (1997).

Mitchell, P., Nature Biotechnology, 20, p-225 (2002).

Zhu, H., et. al., Current Opinion in Chemical Biology, 7, p-55 (2003).

Kim, J. H., et al, Journal of Colloid and Interface Science, 227, p-247 (2000).

Oh, S. J., Langmuir, 18, p-1764 (2002).

Heller, M. J., et al, Integrated Microfabricated Biodevices, Marcel and Decker, NY, (2001).

Rowland, R. J., Marcoux, P, Chapters 2 and 7 in Applied Surface Mount Technology: A guide to surface mount materials and processes, Van Nostrand Reinhold, New York (1993).

Ehrat, M., et. al., Chimia, 55,#1,2, p-35 (2001).

Gad-el-Hak, M. editor, The MEMS Handbook, CRC Press, Boca Raton, Fla., (2002).

1. An integrated biochip comprising of a monolithic assembly of tiles of different character, wherein the said “different character” arises from at least one of
a. surface activation,
b. surface morphology,
c. shape,
d. size,
e. material composition and
f. Utility

2. An integrated biochip as in claim 1 for microarray application, wherein probe is placed on the tile surface after the tiles are assembled.

3. An integrated biochip as in claim 2, wherein one or more probe array elements are placed on each tile.

4. An integrated biochip as in claim 3 where the probe elements are placed on the tiles by one of;
a. dispensing from a fluid media,
b. synthesis

5. An integrated biochip as in claim 1, where the tile surface activation is carried out by a treatment where the said treatment results in a change in surface chemistry.

6. An integrated biochip as in claim 5 where the surface treatment is done by at least one of silane treatment and plasma treatment.

7. An integrated biochip as in claim 1 for microarray application where the tile surface morphology is selected from of planar, porous and textured.

8. An integrated biochip as in claim 1 where the monolithic assembly is formed by anchoring the tiles to an underlying substrate.

9. An integrated biochip as in claim 8 which is used for microarray application.

10. A method of manufacturing of an integrated biochip using an automated equipment wherein the said equipment comprises of a tile placement system, wherein the said placement system can place more than 100 tiles/hour to assemble the said Integrated Biochip.

11. A method of manufacturing of an integrated biochip as in claim 10 where the rate of tile assembly is greater than 1,000 tiles/hour.

12. A method of manufacturing of an integrated biochip as in claim 10 where the rate of tile assembly is greater than 10,000 tiles/hour.

13. A method of manufacturing of an integrated biochip as in claim 10 where the rate of tile assembly is greater than 100,000 tiles/hour.

14. A method of manufacturing of an integrated biochip as in claim 10, wherein the Integrated Biochip comprises of an underlying substrate on which the tiles are anchored, and the manufacturing method of making the said Integrated Biochip comprises of following actions;
a. dispensing of adhesive,
b. placing tiles on the said adhesive and
c. curing said adhesive

15. A method of manufacturing of an integrated biochip as in claim 14 where the said adhesive is dispensed on the surface of underlying substrate for all the tiles before initiating the process of placement of the tiles.

16. A method of manufacturing of an integrated biochip as in claim 15 where one of the following process sequence is used;
a. to cure the adhesive after all the tiles are placed and
b. sequential placement of tiles and curing of adhesive is conducted in more than one step, wherein each step comprises of placement of less than all the tiles and curing adhesive only in the area where the tile placement was conducted in the said each step.

17. A method of manufacturing of an integrated biochip as in claim 14 wherein the placement of each tile is preceded by dispensing of the adhesive locally on the surface of the underlying substrate only in a position where the said tile would be placed.

18. A method of manufacturing of an integrated biochip as in claim 17, where one of the following process sequence is used;
a. to cure the adhesive after all the tiles are placed and
b. sequential curing and placement of tiles is conducted in more than one step, wherein each step comprises of placement of less than all the tiles and curing adhesive only in the area where the tile placement was conducted in the said each step.

19. A method of manufacturing of an integrated biochip as in claim 10 where tiles of different character are assembled, wherein the said “different character” arises from at least one of
a. surface activation,
b. type of attached probe,
c. surface morphology,
d. shape,
e. size,
f. material composition and
g. Utility

20. A method of manufacturing of an integrated biochip as in claim 19, where the integrated biochip is a microarray.

21. A method of manufacturing of an integrated biochip as in claim 10 wherein the tile placement system comprises of an element manufactured by micromachining.

22. A method of manufacturing an integrated biochip wherein the tiles are assembled onto a surface of underlying substrate in the following sequence;
a. Dispensing adhesive on surface of underlying substrate in an area to be occupied by first set of tiles,
b. Dispensing tiles on the surface of underlying substrate so that at least one tile is captured at the location of the dispensed adhesive,
c. Curing the adhesive to anchor the tiles on to the surface of underlying substrate,
d. Removing any of the tiles which are not anchored,
Repeating several times, the above sequence with a different set of tiles in different locations on the surface of the underlying substrate until the assembly is complete.

23. A method of manufacturing an integrated biochip as in claim 22 where the biochip is a microarray.

24. A method of manufacturing an integrated biochip as in claim 22 wherein the tiles in different locations on the surface of the underlying substrate may be differentiated by at least one of surface activation and type of probe.

25. A method of manufacturing an integrated biochip as in claim 22 where more than 100 locations on the surface of the underlying substrate are assembled per hour.

26. A method of manufacturing an integrated biochip as in claim 22 where more than 1,000 locations on the surface of the underlying substrate are assembled per hour.

27. A method of manufacturing an integrated biochip as in claim 22 where more than 10,000 locations on the surface of the underlying substrate are assembled per hour.

28. A method of manufacturing an integrated biochip as in claim 22 where more than 100,000 locations on the surface of the underlying substrate are assembled per hour.

29. A method of manufacturing an integrated biochip as in claim 22, where more than one tile is captured on each location on the surface of underlying substrate.

30. A method of manufacturing an integrated biochip as in claim 22, where the tiles are dispensed on the surface of underlying substrate by dispersing the said tiles in the fluid medium.

31. A method of manufacturing an integrated biochip as in claim 22, where the adhesive is cured to anchor the tiles by at least one of radiation and thermal curing.

32. A method of manufacturing an integrated biochip as in claim 22, where the tiles not anchored to the surface of underlying substrate are removed by one of fluid flow, vacuum and mechanical treatment.
